(19) United States
(12) Patent Application Publication
(10) Pub. No.: US 2008/0145936 A1
(43) Pub. Date: Jun. 19, 2008

(54) Control of ES Cell Self Renewal and Lineage Specification, and Medium Therefor

(75) Inventors: Austin Gerard Smith, Edinburgh (GB); Qi-Long Ying, Edinburgh (GB)

Correspondence Address: STERNE, KESSLER, GOLDSTEIN & FOX P.L.L.C., 1100 NEW YORK AVENUE, N.W., WASHINGTON, DC, 20005, US

(73) Assignee: University of Edinburgh, Edinburgh (GB)

(21) Appl. No.: 11/970,377

(22) Filed: Jan. 7, 2008

Related U.S. Application Data
(63) Continuation of application No. 10/513,675, filed on May 10, 2005, now abandoned.

(30) Foreign Application Priority Data
May 8, 2002 (GB) 0210539.3

(51) Int. Cl. C12N 15/00; C12N 5/02

(52) U.S. Cl. 435/455; 435/375

(57) ABSTRACT

Self renewal of pluripotent cells in culture is promoted using a combination of an activator of a signalling pathway downstream of a receptor of the TGF-β superfamily and an activator of a gp130 downstream signalling pathway.

The present invention relates to culture conditions and methods of culturing pluripotent stem cells in order to promote stem cell self renewal and to prevent or control differentiation of the stem cells. The invention further provide's methods for isolating and maintaining homogeneous preparations of pluripotent stem cells. The methods and compositions provided are suitable for culturing and isolating pluripotent stem cells such as embryonic stem (ES) cells.

The establishment and maintenance of in vitro pluripotent stem cell cultures in the presence of medium containing serum and Leukaemia Inhibitory Factor (LIF) is well known (Smith et al. (1988) Nature 336: 688-90). Such methods have been used to maintain pluripotent embryonic stem (ES) cells from strains of permissive mice over many passages. Maintenance and self renewal of pluripotent stem cell cultures is further supported where the stem cells are cultured in the presence of feeder cells or extracts thereof, usually mouse fibroblast cells. Under such conditions it is possible to maintain human ES cells in a pluripotent state over many passages in culture.

A continuing problem in this field is that, despite intense efforts, it remains the case that pluripotent cultures of ES cells can be derived and maintained for extended periods only from a few species and even in those species not from all embryos. In some cases, pluripotent cells can be identified but can not then be maintained in culture for sufficient time to enable study of the cells or their genetic manipulation. This is particularly the case for rodent (other than some strains of mice) cells.

A further problem is that ES cells that can indeed be maintained in a pluripotent state in culture over many passages can only be so maintained using medium that contains serum or serum extract, and hence is undefined, or using cell culture conditions that require the presence of other cells, such as the fibroblast feeder cells used to maintain human ES cells. However, where ES cells are intended to be subjected to subsequent controlled differentiation into desired cell types, it is undesirable to utilise an undefined culture medium or to have heterologous cells present.

The serum typically used in culturing pluripotent stem cells is fetal calf (bovine) serum, which is known to contain a complex mixture of cytokines and other signalling molecules. In order to control differentiation pathways it is undesirable to introduce unknown cytokines to the culture medium whose influence on the eventual outcome of differentiation is unquantifiable, and could be potentially deleterious. Further, each serum batch is unique and introduces variation into culture protocols.

As a result, the ES cells obtained by culture in such complex media, and any differentiated progeny thereof, risk being contaminated by components of the media and/or by cells such as feeder cells that are required to maintain the ES cells. These factors mitigate against development of good manufacturing practices for therapeutic and other applications of ES cells and their progeny.

When deriving a differentiated cell population from an ES cell culture, it is desirable to be able to convert a high proportion of the ES cells into progeny of the same type—i.e. to maintain as homogeneous a population of cells as possible. However, in practice, it is observed that, following differentiation, a cell population is obtained that contains a heterogenous mixture of cells. Hence, it is desirable to be able to carry out differentiation of an ES cell population obtaining a purer population of progeny.

EP 1077254 describes methods and compositions for the differentiation of stromal cells from adipose tissue, which may include interleukins, FGF and serum, and amounts of TGF-β sufficient to induce differentiation into smooth muscle.

EP 0753574 describes methods and compositions for ex vivo human progenitor cell expansion. The culture medium contains stromal cells, typically transformed fibroblast cells.

WO 00/05344 describes maintenance of Drosophila germline stem cells and propagation of somatic stem cells of other species when co-cultured with genetically engineered Drosophila cells.

WO 96/40866 describes serum-free culture of human haematopoietic progenitor and stem cells in a culture medium containing at least one of a peptone, a protease inhibitor and a pituitary extract.

US 2002/0028510 describes methods and compositions for the differentiation of pluripotent cells from umbilical cord blood into neuronal cell types.

U.S. Pat. No. 5,750,376 describes methods and compositions for differentiation of multipotent neural stem cells in culture medium supplemented with at least one growth factor.

Wiles and Johansson, Exp. Cell Research, 1999 (247) pgs 241-248 describes maintenance of undifferentiated cell lines in the presence of LIF and Fetal Bovine Serum. When ES cells were grown in a serum free medium they rapidly lost their ES cell phenotype and developed into a range of cell types, including neuroectoderm.

Hence, an object of the invention is to provide methods of culturing and culture media suitable for pluripotent stem cells, which are capable of supporting self-renewal of said stem cells in an undifferentiated state for many passages. A further object of the invention is to provide a culturing system that permits maintenance of a pluripotent stem cell culture in vitro until differentiation of the cells is induced in a controlled manner. A still further object of the invention is to provide methods and compositions that enhance the isolation of pluripotent stem cells and facilitate their isolation from organisms refractory to ES cell isolation or from which pluripotent stem cells have not yet been isolated.

The present invention is based on the observation that culturing pluripotent stem cells, such as ES cells, in serum-free media comprising agonists of the gp130 (e.g. LIF) and TGF-β superfamily (e.g. BMP4) signalling pathways promotes self renewal of the stem cells for multiple passages. Hence, in the presence of gp130 signalling, an agonist of the TGF-β superfamily signalling pathway provides a self renewal stimulus rather than a pro-differentiation signal.

Accordingly, a first aspect of the invention provides the use of a combination of:
(a) an activator of a signalling pathway downstream from a receptor of the TGF-β superfamily; and
(b) an activator of a gp130 downstream signalling pathway, in promoting self-renewal of pluripotent cells in culture.

One or more signalling pathways downstream from a receptor of the TGF-β superfamily can be activated using an agonist of a receptor of the TGF-β superfamily. Examples of receptors are the BMP receptor and the TGF-β receptor, as well as receptors bound by activin and nodal. Activation of one or more signalling pathways is preferably achieved using an agonist of a BMP receptor.

Activation of one or more gp130 downstream signalling pathways can be achieved by use of a cytokine acting through gp130, for example a cytokine or other agonist of the LIF receptor.

An agonist of a receptor of the TGF-β superfamily is suitably a member of the TGF-β superfamily of signalling factors, and is preferably a bone morphogenetic protein (BMP) such as BMP4. Known homologues of BMP4, such as BMP2 and BMP7, are also suitable, as are homologues from non-mammalian species such as decapentaplegic (dpp) from Drosophila melanogaster. Good results have been obtained using BMPs 4, 8 and 2. Good results have also been obtained using a chimera of BMP4 and BMP8, which is easier to obtain in secreted form than BMP4. The term “agonist” is also intended to embrace mimetics, fusion proteins or chimaeras of TGF-β superfamily signalling polypeptides, and fragments, variants and derivatives thereof, capable of activating receptors of the TGF-β superfamily.

Cytokines capable of acting through gp130, and thus of activating gp130 signal transduction, include LIF, CNTF, cardiotrophin, oncostatin M and IL-6 plus sIL-6 receptor. Suitable cytokines include mimetics, fusion proteins or chimaeras that can bind to and/or activate signalling though gp130.

The role of cytokines acting through gp130 in the presence of serum is well established, but the capacity of those cytokines to sustain undifferentiated cells in the absence of serum is limited.

Previous data on BMPs, both from developmental studies in vivo and in ES cells, points to activity in driving mesodermal and epidermal differentiation. There is no evidence in the literature that activation of the BMP signalling pathway can enhance self-renewal in pluripotent stem cells.

Chemically defined serum-free media for ES cell differentiation comprising LIF and BMP4 has been previously disclosed by Johansson and Wiles (Mol. Cell. Biol. (1995) 15(1): 141-51). However, the medium also contained an undefined albumin component and both LIF and BMP4 were only combined in a culture medium used for promoting differentiation of ES cells into mesoderm and haematopoletic cells. Differentiation into cells expressing the mesodermal marker gene, Brachyury, was observed after 5 days in culture (see page 145, first paragraph). In the absence of BMP4, the chemically defined medium supplemented with much higher concentrations of LIF is capable of maintaining ES cells for a maximum of three passages (see page 143, second column). Hence, Johansson and Wiles describe a composition which promotes differention induced by formation of multicellular aggregates and differentiation of ES cells, not self renewal of ES cells.

The present invention therefore provides, in one embodiment, culture of ES cells in medium that may be free of serum, serum extract, feeder cells and feeder cell extract.

When using the LIF and BMP4 -containing medium of a specific embodiment of the present Invention extended passaging of ES cells is possible. In one example, murine ES cells have been maintained for more than 20 passages over a three month period. Cells were passaged every 2-4 days depending on the plating cell density, though occasionally cells were passaged 7-10 days after plating at low density.

Another advantage of the present culture system is that differentiation of ES cells is reduced compared to culture in the presence of serum. This is significant because often the most pluripotent ES cells tend to differentiate considerably in serum, making their manipulation and expansion problematic.

ES cells in the cultures were examined and found still to maintain ES cell morphology and to express ES cell markers (positive staining for alkaline phosphatase). When using a GFP reporter gene under the control of the ES cell specific Oct-4 promoter green fluorescence was seen in the ES cells. Similarly, β-galactosidase expression from the Sox2 promoter was maintained. ES cell mRNA markers such as Oct4, Nanog, Rex1 and Sox2 are expressed, further confirming the cells are ES cells. The morphological differentiation of cells in the culture is found to be very low, and lower than a comparative culture of ES cells in serum-containing medium. Thus, the culture conditions of the present invention enable ES cells to self-renew in the absence of serum.

Embryonic stem cells have been reported from a number of mammalian sources including mouse (Bradley et al (1984) Nature 309: 255-56), American mink (Mol Reprod Dev (1992) Dec;33(4):418-31), pig and sheep (J Reprod Fertil Suppl (1991);43:255-60), hamster (Dev Biol (1988) May;127(1):224-7) and cow (Roux Arch Dev Biol (1992); 201: 134-141). It will be appreciated that the methods and compositions of the present invention are suitable for adaptation to culturing of other mammalian pluripotent cell cultures, including human, primate and rodent, and avian ES cells.

Specifically, with regard to human ES cells, it is known that human ES cells respond to LIF and therefore the medium and methods of the invention, in which a self-renewal stimulus is obtained in response to a combination of LIF and BMP4, are of application to human ES cells.

Suitable cell densities for the methods of the invention will vary according to the pluripotenf stem cells being used and the natures of any desired progeny. Good results have been obtained by culturing embryonic stem cells in monolayer culture, dissociating the embryonic stem cells and subsequently culturing the embryonic stem cells in monolayer culture on a culture surface at a density of from 0.2-2.5×10⁴ cells per cm², more particularly at a density of from 0.5-1.5×10⁴ per cm²; The cells proliferate as adherent monolayers and are observed to have a doubling time comparable to ES cells grown in serum-containing media together with LIF.

Typical surfaces for culture of ES cells and their progeny according to the invention are culture surfaces recognized in this field as useful for cell culture, and these include surfaces of plastics, metal, composites, though commonly a surface such as a plastic tissue culture plate, widely commercially available, is used. Such plates are often a few centimetres in diameter. For scale up, this type of plate can be used at much larger diameters and many repeats plate units used.

It is further common for the culture surface to comprise a cell adhesion protein, usually coated onto the surface. Receptors or other molecules on the cells bind to the protein or other cell culture substrate and this promotes adhesion to the surface and it is suggested promotes growth. Gelatin coated plates are commonly available and are suitable for the invention, and other proteins may also be used.

In an embodiment of the present invention, including an agent that suppresses differentiation, such as an inhibitor of the FGF receptor in the culture medium for at least part of the culturing period is found to suppress the tendency of ES cells to differentiate. In one embodiment, the ES cells are cultured in defined serum-free media comprising LIF and an FGF receptor inhibitor for a specified period before the FGF receptor inhibitor is removed and replaced by an agonist of the BMP signalling pathway (e.g. BMP4). Suitable FGF receptor inhibitors include the compounds SU5402 and PD173074. Alternatively, a competitive inhibitor of the FGF receptor can be used, suitably a soluble form of the receptor.

In an alternative embodiment, it is an option not to remove the FGF receptor inhibitor. Hence, the FGF receptor inhibitor is present in the culture medium for an extended period, either in the presence or absence of an agonist of the BMP signalling pathway. ES-cells can be grown in culture for at least 20 passages in N2B27 medium in the presence of LIF and an FGF inhibitor, in the absence of BMP. If the FGF receptor inhibitor is not removed from the medium, it is preferred that it is a specific inhibitor and has little or no activity on other receptors.

A second aspect of the invention provides a method of culture of ES cells so as to promote ES cell self renewal, comprising maintaining the ES cells in medium containing:
(a) an activator of a signalling pathway downstream from a receptor of the TGF-β superfamily; and
(b) an activator of a gp130 downstream signalling pathway.

In one embodiment, the method of ES cell culture comprises maintaining the ES cells in a medium containing:
(a) an agonist of a receptor of the TGF-β superfamily; and
(b) a cytokine acting through gp130.

Methods of the invention can be used for stimulating self-renewal of ES cells in medium which is free of serum and free of serum extract, which cells have previously been passaged in the presence of serum or serum extract. Preferably, such methods are also carried out in the absence of feeder cells and/or feeder cell extracts. For example, culture of ES cells can be carried out comprising the steps of:
maintaining the ES cells in a pluripotent state in culture, optionally on feeders, in the presence of a cytokine acting though gp130 and serum or an extract of serum;
passaging the ES cells at least once;
withdrawing the serum or the serum extract from the medium and withdrawing the feeders (if present), so that the medium is free of feeders, serum and serum extract; and
subsequently maintaining ES cells in a pluripotent state in the presence of an activator of a signalling pathway downstream from a receptor of the TGF-β superfamily and an activator of a gp130 downstream signalling pathway.

At around the time that the serum or extract of serum is withdrawn from the medium, it is an option to add to the medium an agent that suppresses differentiation, for example, an FGF-receptor inhibitor. It is an option for the inhibitor of differentiation to be withdrawn at the same time as or subsequent to maintenance of the cells in the presence of an activator of a signalling pathway downstream from a receptor of the TGF-β superfamily.

The present invention also provides a method of obtaining a transfected population of ES dells, comprising:
transfecting ES cells with a construct encoding a selectable marker;
plating the ES cells;
culturing the ES cells in the presence of
(a) an activator of signalling pathways downstream from a receptor of the TGF β superfamily; and
(b) an activator of gp130 downstream signalling pathways; and selecting for ES cells that express the selectable marker.

The selectable marker may encode antibiotic resistance, a cell surface marker or another selectable marker as described e.g. in EP-A-0695351.

In a further embodiment, the present invention provides a method of culture of ES cells, comprising the steps of transferring an individual ES cell to a culture vessel, such as an individual well on a plate, and culturing the ES cell in the presence of an activator of signalling pathways downstream from a receptor of the TGF β superfamily and an activator of gp130 downstream signalling pathways, so as to obtain a clonal population of ES cells, all of which are progeny of a single ES cell.

Once a stable, homogenous culture of ES cells is obtained, the culture conditions can be altered to direct differentiation of the cells into one or more cell types selected from ectodermal, mesodermal or endodermal cell fates. Addition of, or withdrawal of cytokines and signalling factors, can enable the derivation of specific differentiated cell populations at high efficiency. Differentiation of an ES cell towards a non-neuroectodermal fate may be achieved by maintaining the ES cell in the presence of a cytokine acting through gp130 and an agonist of the BMP receptor and then withdrawing the cytokine whilst maintaining the BMP receptor agonist and/or adding a further signalling molecule capable of directing differentiation.

For example, exposure to BMP4 in the absence of LIF leads to induction of mesendodermal cell types. Withdrawal of agonists of the gp130 and TGF-β signalling pathways and/or blockade of both pathways leads to induction of a neurectodermal phenotype. Alternatively, other signalling factors can be added to the culture conditions to direct other differentiation pathways—for example, activin, sonic hedgehog (shh), Wnts and FGFs.

In use, towards the end of ES cell culture, it is desirable to remove BMP4 at least one passage before differentiation is initiated, in order to ensure that the BMP signal declines and there is no legacy of the BMP signal during subsequent differentiation. In one embodiment, an FGF receptor antagonist is added to the cultures for one to two passages whilst removing BMP from the cultures.

Further aspects of the invention provide for cell culture media for self-renewal of ES cells. One such medium comprises:
basal medium;
an activator of signalling pathways downstream of a receptor of the TGF-β superfamily;
an activator of gp130 downstream signalling pathways;
an iron-transporter;
   wherein the medium is free of serum and serum extract.

Basal medium is medium that supplies essential sources of carbon and/or vitamins and/or minerals for the ES cells. The basal medium is generally free of protein and incapable on its own of supporting self-renewal of ES cells. The iron transporter provides a source of iron or provides ability to take up iron from the culture medium. Suitable iron transporters include transferrin and apotransferrin.

It is preferred that the medium further comprises insulin or insulin-like growth factor and is free of feeder cells and feeder cell extract.

The invention also provides cell culture media comprising:
(a) an agonist of a TGF-β receptor; and

(b) a cytokine acting through gp130.

The culture medium is optionally supplemented with an inhibitor of differentiation of ES cells, or, when differentiation is desired, signalling factors that direct differentiation of ES cells toward a specific phenotype.

It is preferred that the medium is free of serum or serum extract. Most preferably, the medium is fully defined.

In a preferred embodiment of the invention the culture medium comprises the gp130 receptor binding cytokine, LIF, at a concentration of between 10U/ml and 1000U/ml, more preferably between 50U/ml, and 500U/ml, even more preferably in the region of 100 U/ml.

It will be appreciated by a person of skill in the art that activation of signalling pathways downstream from a receptor of the TGF-β superfamily can be effected by either upstream agonists of the TGF-β receptor (e.g. receptor ligands), constitutively active receptors, or activated downstream components of the signalling pathway, for example the SMAD signal transduction molecules. Likewise upstream effectors (eg. cytokines) and downstream effectors (eg. Stats) of the gp130signal transduction pathway are capable of activating this pathway also. Thus, the present invention embraces all compositions comprising molecules capable of activating TGF-β receptor superfamily signalling pathways and gp130 signalling pathways in order to promote self renewal of pluripotent stem cells.

It is further preferred, according to the invention, that culture of cells is carried out in an adherent culture, and in examples of the invention it has been found that following maintenance of cells in a pluripotent state, differentiation can be induced with a high degree of uniformity and with high cell viability. Adherent cultures may be promoted by the inclusion of a cell adhesion protein, and in specific examples of the invention gelatin has been used as a coating for the culture substrate.

It is also preferred to culture pluripotent cells according to the invention in monolayer culture, though it is optional for cells to be grown in suspension culture or as pre-cell aggregates; cells can also be grown on beads or on other suitable scaffolds such as membranes or other 3-dimensional structures.

A further component of medium for culture of pluripotent cells according to the invention, and which is preferred to be present, is a factor promoting survival and/or metabolism of the cells. In a specific embodiment of the invention, cells are cultured in the presence of insulin. An alternative factor is insulin-like growth factor and other such survival and/or metabolism promoting factors may alternatively be used.

Culture medium used in the examples of the invention preferably also comprises serum albumin. This can be used in purified or recombinant form, and if in a recombinant form this has the advantage of absence of potential contaminating factors, cytokines etc. The culture medium does not need to contain serum albumin and this component can be omitted or replaced by another bulk protein or by a synthetic polymer (polyvinyl alcohol) as described by Wiles et al.

A particularly preferred medium of the invention is one that is fully defined. This medium does not contain any components which are undefined, that is to say components whose content is unknown or which may contain undefined or varying factors that are unspecified. An advantage of using a fully defined medium is that efficient and consistent protocols for culture and subsequent manipulation of pluripotent cells can be derived. Further, it is found that maintenance of cells in a pluripotent state is achievable with higher efficiency and greater predictability and that when differentiation is induced in cells cultured using a defined medium the response to the differentiation signal is more homogenous then when undefined medium is used.

A medium according to the present invention may be used for culture of pluripotent stem cells from any adult tissue.

Methods of the invention also include a method of obtaining a differentiated cell comprising culturing a pluripotent cell as described and allowing or causing the cell to differentiate, wherein the cell contains a selectable marker which is capable of differential expression in the desired differentiated cell compared with other cell-types, including pluripotent stem cells, whereby differential expression of the selectable marker results in preferential Isolation and/or survival and/or division of the desired differentiated cells.

The differentiated cell can be a tissue stem or progenitor cell, and may be a terminally differentiated cell.

The present invention also provides a method of isolating a pluripotent stem cell or an EG or EC cell comprising culturing tissue from an embryo, fetus or adult in medium containing:
(a) a cytokine acting through gp130; and
(b) an agonist of a TGF β receptor; and/or
(c) an inhibitor of a FGF receptor

Preferably, the medium is a fully defined medium.

In a further embodiment, the invention provides use of a combination of:
(a) an inhibitor of FGF receptor signalling; and
(b) an activator of gp130 downstream signalling pathways in promoting self-renewal of pluripotent cells in culture.

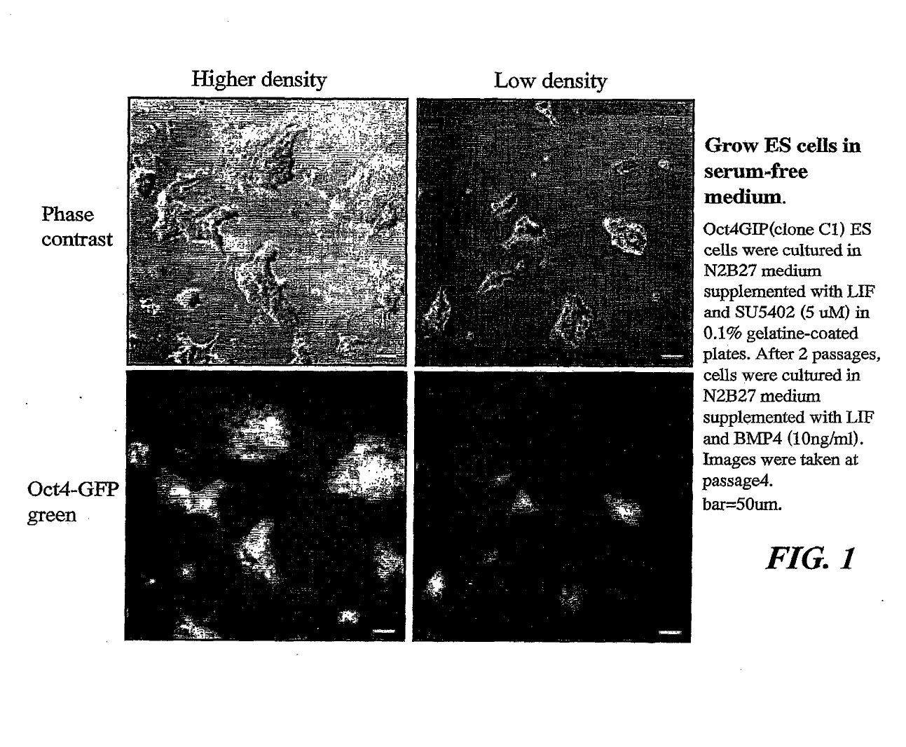

The methods and compositions of the invention are illustrated in the accompanying drawing in which:
FIG. 1 shows a light microscopy image of ES cells grown at either high or low cell density in the serum-free defined media of the invention; cells expressing the GFP protein under the control of the ES cell specific promoter, Oct4, fluoresce;

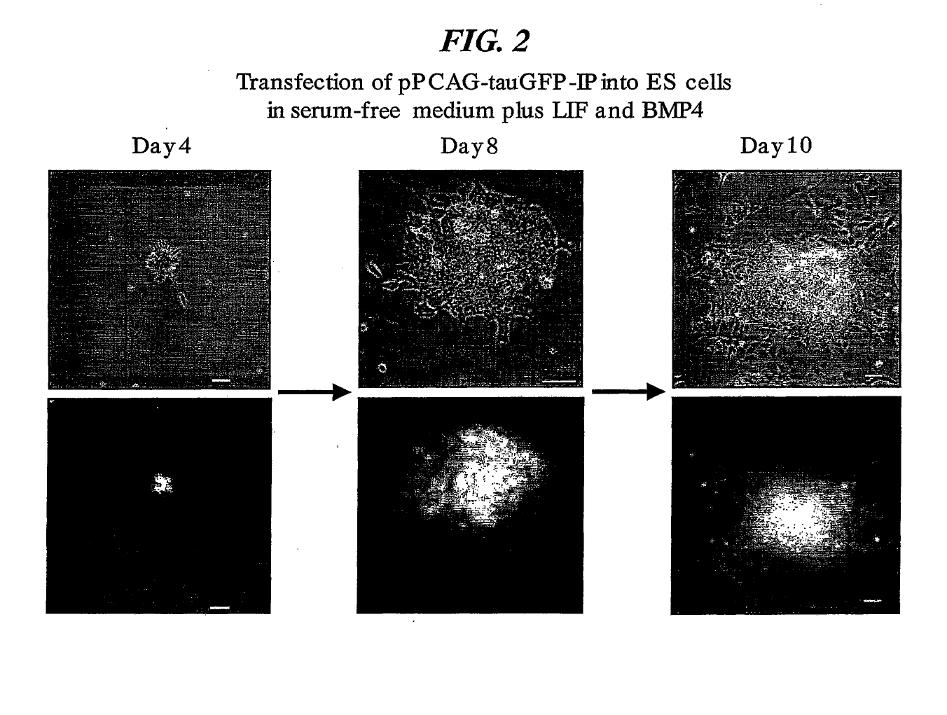

FIG. 2 shows light microscopy images of the ES cells stably transfected with pPCAG-tauGFP-IP in serum-free medium in the presence of LIF and BMP4;

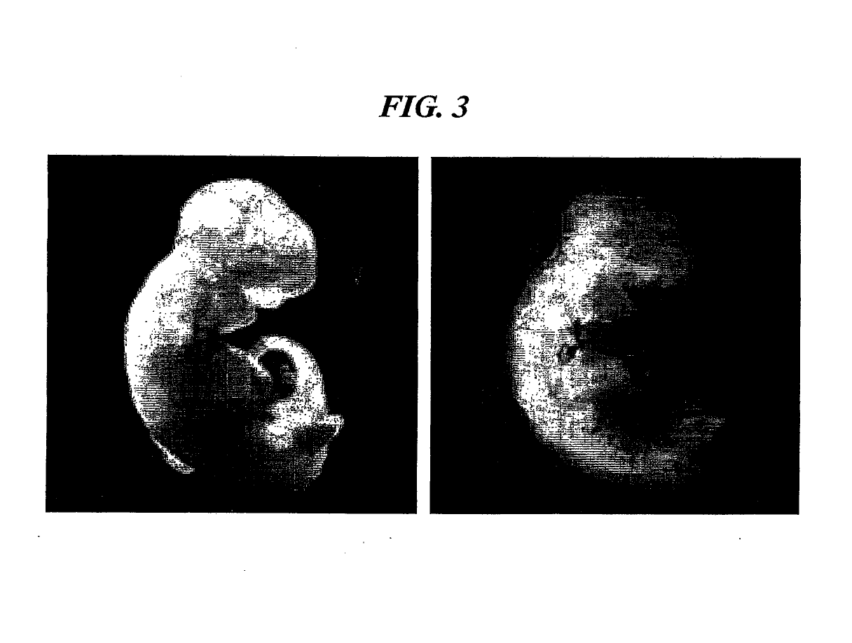

FIG. 3 shows a chimeric mouse embryo (at embryonic day 11) created from blastocysts injected with GFP-expressing ES cells of FIG. 2;

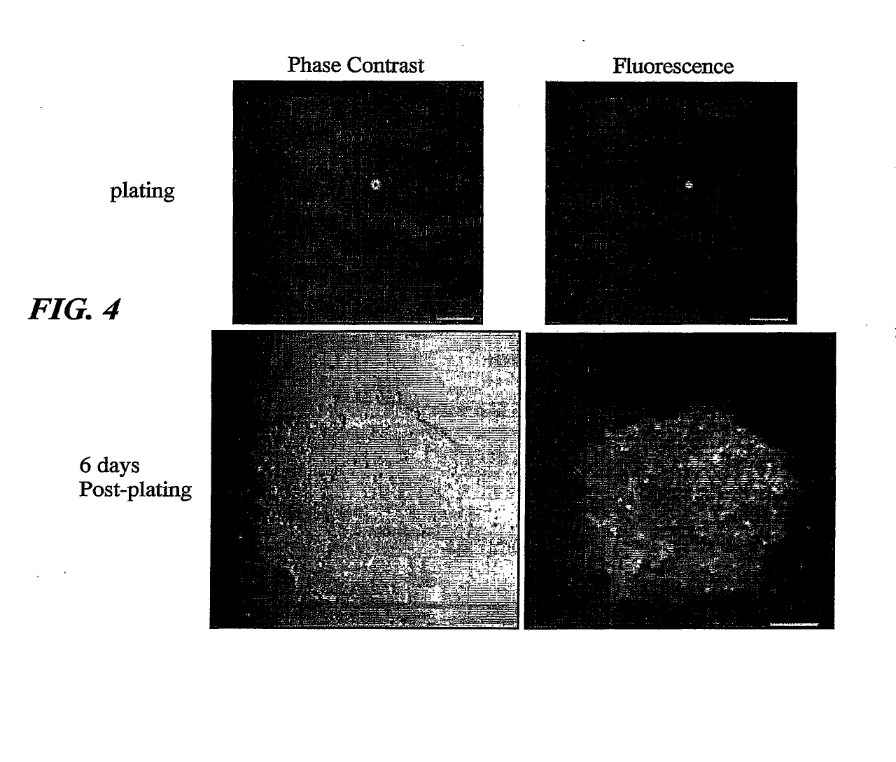

FIG. 4 shows phase contrast and fluorescence images of individual ES cells plated at low density in the presence of LIF and BMP4, and clonal populations derived therefrom at 6 days post-plating;

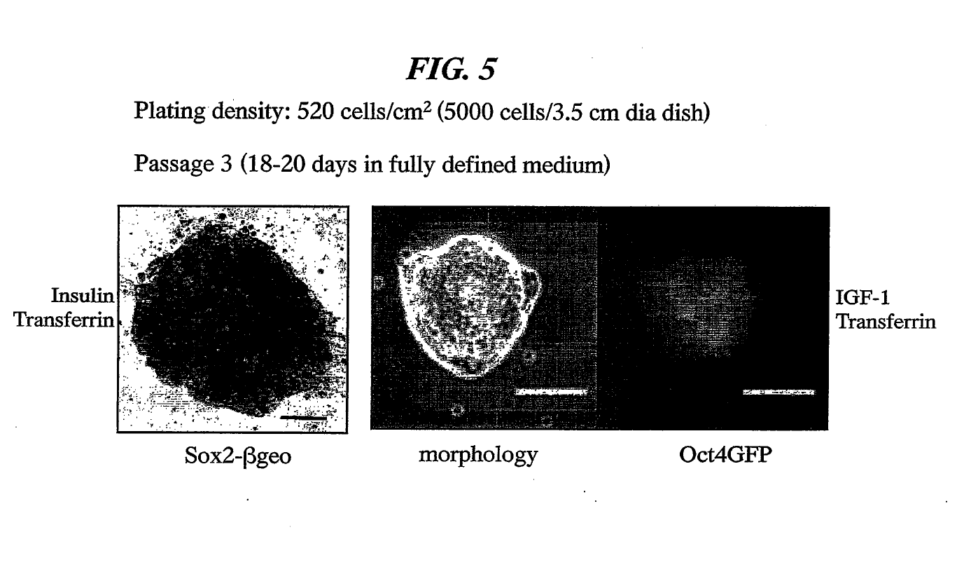

FIG. 5 shows OS25 and Oct4GFP ES cells (passage 3) grown in DMEM F12 plus neurobasal medium (ratio 1:1) supplemented with IGF-1 or insulin, apotransferrin, progesterone, putrescine and sodium selenite;

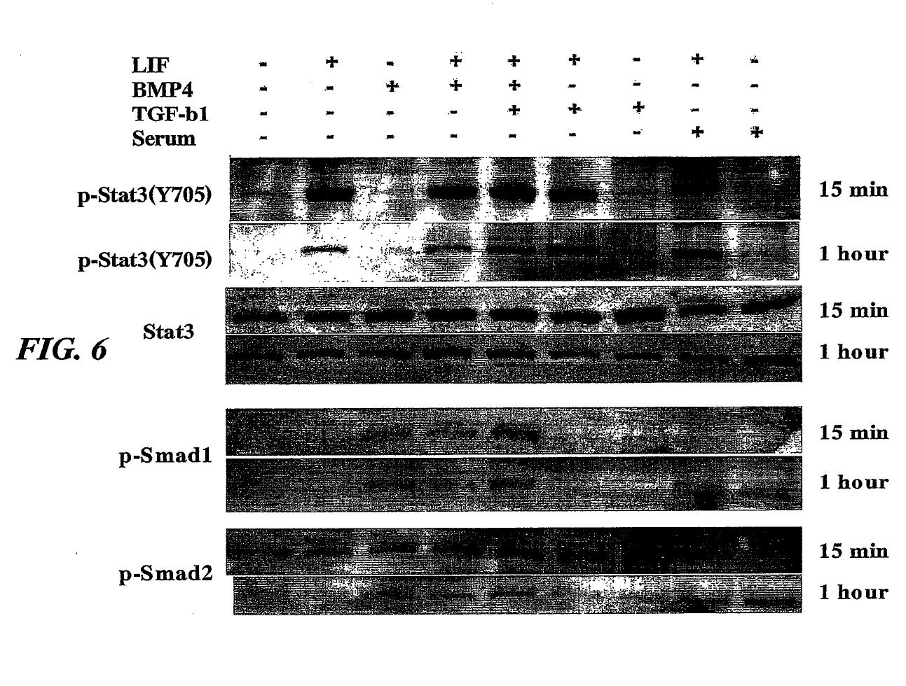

FIG. 6 shows an immunoblot demonstrating phosphorylation of p-STAT3, STAT3, p-Smad1 and p-Smad2 at 15 min and 1 hour post administration of one or more of LIF/BMP4/TGF-β1/serum to ES cells; and

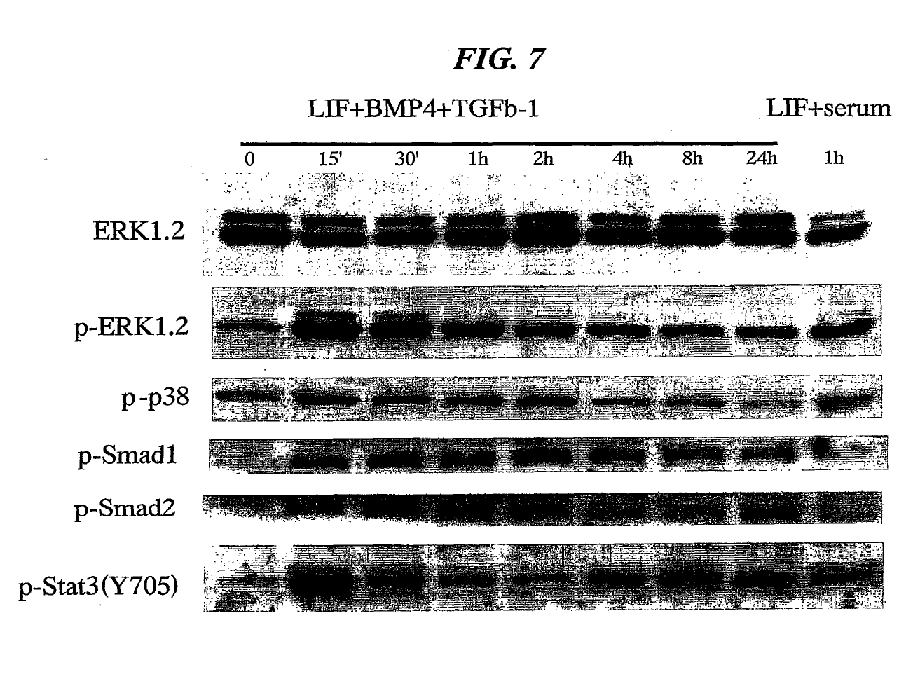

FIG. 7 shows an immunoblot demonstrating phosphorylation of ERKs, p38, Smads1 and 2 and STAT-3 at 0, 15 mins, 30 mins, 1, 2, 4, 8 and 24 hours post administration of LIF plus BMP4 plus TGF-β1 to ES cells. The phosphorylation pattern at 1 hour post-administration is compared with that 1 hour post-administration of LIF+serum.

The invention is demonstrated in use by the following examples.

### EXAMPLES

### Example 1. Method for Growing ES Cells in Serum Free, Feeder Cell Free Defined Culture Media

N2B27 (1:1 mixture of DMEM/F12 medium supplemented with modified N2 (25 μg/ml insulin, 100 μg/ml apo-transferrin, 6 ng/ml progesterone, 16 μg/ml putrescine, 30 nM sodium selenite and 50 μg/ml bovine serum albumin) and Neurobasal medium supplemented with B27)

ES cells were cultured in 0.1% gelatin coated dishes in N2B27 medium supplemented with LIF (100U/ml) and BMP4 (10 ng/ml). For passaging, a standard protein-free cell dissociation buffer was used to dissociate cells.

The plating density of the cells was approximately 1-5×10⁴/cm².

At the start of culture, the medium was further supplemented with SU5402 (5 μM) to suppress differentiation. Cells were transferred to media free of SU5402 after two passages.

ES cells were maintained in these serum free conditions for 20 passages over a three month period. Cells were normally passaged every 2-4 days depending on plating density. Occasionally, cell were passaged 7-10 days after plating at low clonal density.

The ES cells maintained pluripotency after multiple passages. Upon withdrawal of LIF and BMP4 the ES cells differentiated into Sox1 expressing neural precursor cells. In the presence of BMP4 but absence of LIF, the ES cells differentiated into large flat cells of characteristic morphology.

### Example 2. Oct4-GFP Expression is Maintained in ES cells Cultured Under Serum Free Conditions

Oct4GFP (clone C1) ES cells were cultured in N2B27 medium supplemented with LIF and SU5402 (see Example 1) in 0.1% gelatine coated plates. After two passages cells were cultured in N2B27 medium supplemented with LIF and BMP4. After a further two passages light microscope images were taken of the cells under phase contrast to show morphology and UV fluorescence to show expression of GFP. The microscopy images are shown in FIG. 1.

It is apparent that both the morphology and expression of GFP indicates that after four passages in serum free media the ES cells maintained their pluripotent phenotype.

### Example 3. Stable Transfection of ES Cells

E14 TG2A ES cells were cultured in DMEM F12 plus neurobasal medium supplemented with N2-B27 additives and also supplemented with LIF and BMP4. The cells were propagated on 0.1% gelatin coated plates, harvested and electroporated with pPCAG-tauGFP-IP. Transfected cells were replated on a 10 cm. diameter dish at a density of 10⁵-10⁶ per dish. After 24 hours, 0.5 μg/ml puromycin was added to select for positive colonies.

Between 8 and 10 days later, single GFP positive colonies were picked into each single well of a 96 well plate and the cells cutured in the same medium as described above.

Stable transfection of the ES cells by GFP fluorescence and expansion of morphologically undifferentiated ES cells was observed, as demonstrated in FIG. 2.

### Example 4. Chimeric Mouse

GFP expressing ES cells obtained as described in Example 3 above were injected into blastocysts. GFP expressing ES cells contributed to a wide range of cell-types in a chimeric mouse embryo, as shown in FIG. 3 (embryonic day 11). Live born chimeric mice were obtained, having a chimeric coat, confirming that the cells were true ES cells.

### Example 5. Clonal Self-renewal of ES Cells

Individual ES cells were picked and transferred into wells of a 96 well plate, and cultured in medium as described for Example 3 above.

We found the efficiency of cloning of these ES cells, previously grown in serum-free media for at least one passage, to be similar to that obtainable using serum-containing medium, i.e. about 10% efficiency. The clones grew and could be passaged and grown further as undifferentiated ES cells in the presence of LIF and BMP4, as shown in FIG. 4.

In previous experiments (data not published) we have discovered that ES cells grown in serum-containing medium when transferred directly to a serum-free medium demonstrate lower formation of clonal colonies.

### Example 6. Growth of ES Cells in Fully Defined Medium

ES cells were grown in a fully defined, albumin free, medium comprising DMEM F12 plus neurobasal medium (ratio 1:1) supplemented with IGF-1 10 μg/ml (or insulin at 5-25 μg/ml), apotransferrin 100 μg/ml, progesterone 6 μg/ml, putrescine 16 μg/ml and sodium selenite 30 nmol.

Oct4GFP ES cells were passaged 6 times (cells passaged every 6-8 days) using cell dissociation buffer and replated after each passage at low density. Microscopy images taken after passage 3 are shown in FIG. 5.

### Example 7. Use of Serum-Free Medium and Transient Growth Factor Stimulation

ES cells were grown initially in a medium comprising DMEM F12 plus neurobasal medium (ratio 1:1) supplemented with N2-B27. ES cells were plated at very low density, about 1000-10,000 cells on a 3.5 cm diameter plate and grown on in the same medium supplemented with LIF and BMP4.

Intermittently, TGFβ at 1-2 ng/ml or activin A at 5 ng/ml were added to the culture medium and the cells grown on again.

We observed enhanced numbers of undifferentiated ES cells, indicating increased proliferation or increased ES cell survival, or both. Hence the rank order of efficiency was found to be:

LIF/BMP4/activin A (5 ng/ml)>LIF/BMP4/TGF-β (1-2 ng/ml)>LIF/activin A >LIF/BMP4>LIF/TGF-β1>LIF only.

After more (5-6) passages the rank order of efficacy changed to:

LIF/BMP4>LIF/BMP4/activin A>LIF/BMP4/TGF-β>LIF/activin A=LIF only.

When either TGFβ or activin A was continuously present, progressive differentiation of the ES cells was observed.

Administration of LIF and BMP4 was observed to lead to phosphorylation of SMADs, ERK and STAT3 (see FIGS. 6 and 7)—demonstrating that there is activation of pathways downstream from the BMP and gp130 receptors.

### Example 8. Comparison of ES Cells Grown in Serum-free and Serum-containing Medium.

We cultured Oct4GFP ES cells in 0.1% gelatin coated 6 well plates at a density of 4×10⁶ cells per well in the following media: N2B27 plus LIF, N2B27 plus LIF plus BMP4 and GMEM 10% FCS plus LIF. The results are shown in Table 1. Cell dissociation buffer was used to dissociate cells. After passage 5 the number of cells grown in N2B27 plus LIF was not sufficient to continue culture.

The invention thus provides medium and methods for self-renewal of ES cells of many species.

TABLE
Comparison of ES cells grown in serum-free and serum-containing medium

| | N2B27 + LIF | | N2B27 + LIF + BMP4 | | GMEM/10% FCS + LIF | |
|---|---|---|---|---|---|---|
| | Fold change | Oct4GFP % | Fold change | Oct4GFP % | Fold change | Oct4GEP % |
| P1 | 3.1 ± 0.4 | 93.7 ± 0.8 | 2.4 ± 0.3 | 93.3 ± 0.8 | 2.4 ± 0.3 | 92.4 ± 2.4 |
| P2 | 2.3 ± 0.5 | 96.1 ± 1.6 | 2.2 ± 0.3 | 95.5 ± 1.0 | 1.8 ± 0.3 | 96.8 ± 0.4 |
| P3 | 3.5 ± 0.7 | 95.0 ± 2.2 | 2.7 ± 0.5 | 93.5 ± 2.8 | 3.3 ± 0.7 | 94.0 ± 1.9 |
| P4 | 2.2 ± 0.5 | 91.4 ± 2.4 | 3.6 ± 0.8 | 95.7 ± 1.2 | 1.7 ± 0.2 | 90.3 ± 0.9 |
| P5 | 1.3 ± 0.4 | 95.3 ± 2.6 | 2.4 ± 0.2 | 97.7 ± 1.8 | 1.9 ± 0.2 | 95.1 ± 3.7 |
| P6 | | | 3.2 ± 0.3 | 97.2 ± 1.0 | 1.5 ± 0.3 | 94.2 ± 2.4 |
| P7 | | | 3.9 ± 1.0 | 97.4 ± 0.4 | 2.0 ± 0.5 | 86.0 ± 4.7 |
| P8 | | | 3.4 ± 0.2 | 95.7 ± 0.6 | 2.1 ± 0.3 | 91.9 ± 6.7 |

Oct4GIP ES cells were cultured in 0.1% gelatin-coated 6-well plates at a density of 4 × 10⁵ cells/well in the medium as indicated. Cell dissociation buffer was used to dissociate cells. After passage 5, the number of cells grown in N2B27 + LIF was not enough for continuing culture.

1. A method of self-renewing pluripotent cells in culture, comprising maintaining the cells in medium containing:
(a) a bone morphogenetic protein (BMP); and
(b) an agonist of a leukemia inhibitory factor (LIF) receptor selected from (i) LIF, and (ii) a combination of IL-6 and soluble IL-6 receptor, whereby the cells proliferate without differentiating.

2-3. (canceled)

4. The method of claim 1, wherein the BMP is BMP4.

5. (canceled)

6. The method of claim 1, wherein the agonist is LIF.

7. The method of claim 1, wherein the agonist is soluble IL-6 in combination with soluble IL-6 receptor.

8. (canceled)

9. The method of claim 1, wherein the cells are cultured in the presence of a FGF receptor inhibitor.

10. The method of claim 9 wherein the FGF receptor inhibitor is SU5402 or PD173074.

11. (canceled)

12. The method of claim 1, wherein the cells are pluripotent cells selected from mammalian and avian pluripotent cells.

13. The method of claim 1 wherein the cells are ES cells.

14. The method of claim 1, wherein the cells are selected from rodent, bovine, porcine, ovine and primate pluripotent stem cells.

15. (canceled)

16. The method of claim 1, wherein the cells are selected from rat cells and mouse cells.

17. The method of claim 1, wherein the cells are human cells.

18. The method of claim 1, comprising maintaining the cells in medium that is free of serum, free of serum extract, free of feeder cells and free of feeder cell extract.

19. The method of claim 1, wherein the medium is a fully defined medium.

20. A method of culture of ES cells, comprising maintaining the ES cells in medium containing:
(a) a BMP; and
(b) an agonist of a LIF receptor selected from (i) LIF, and (ii) a combination of IL-6 and soluble IL-6 receptor.

21-23. (canceled)

24. The method of claim 20, wherein the agonist is LIF.

25. The method of claim 20, wherein the medium further comprises an inhibitor of a FGF receptor.

26. A method of culture of ES cells, comprising:
maintaining the ES cells in a pluripotent state in culture, in the presence of an agonist of a LIF receptor selected from (i) LIF, and (ii) a combination of IL-6 and soluble IL-6 receptor, and serum or an extract of serum, optionally on feeders;
passaging the ES cells at least once;
withdrawing the serum or the serum extract from the medium and withdrawing the feeders if present, so that the medium is free of feeders, serum and serum extract; and
subsequently maintaining ES cells in a pluripotent state in the presence of
(a) a BMP; and
(b) an agonist of a LIF receptor selected from (i) LIF and (ii) a combination of IL-6 and soluble IL-6 receptor.

27. The method of claim 26, comprising culturing the ES cells in the presence of a FGF receptor inhibitor.

28. The method of claim 27 wherein the inhibitor is added to culture medium at the time that serum or serum extract is withdrawn.

29. The method of claim 28 wherein at the same time as or subsequent to maintenance of the cells in the presence of a BMP, the inhibitor is withdrawn.

30. A method of obtaining a transfected, pluripotent population of ES cells, comprising:
transfecting ES cells with a construct encoding a selectable marker;
plating the ES cells;
culturing the ES cells in the presence of
(a) a BMP;
(b) an agonist of a LIF receptor selected from (i) LIF, and (ii) a combination of IL-6 and soluble IL-6 receptor; and
selecting for ES cells that express the selectable marker thereby obtaining transfected, pluripotent ES cells.

31. The method of claim 30 wherein the selectable marker encodes antibiotic resistance or a cell surface marker.

32-51. (canceled)
